(19) United States
(12) Patent Application Publication
(10) Pub. No.: US 2008/0251758 A1
(43) Pub. Date: Oct. 16, 2008

(54) Triazine Compounds Comprising Substituents Containing Amino Groups and Carboxyl Groups

(75) Inventors: Jochen Kirchhoff, Luedinghausen (DE); Peter Werle, Gelnhausen (DE); Guenter Kreilkamp, Marl (DE); Edeltraud Kruppa, Herten (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Evonik Degussa Gmbh, Essen (DE)

(21) Appl. No.: 12/090,541

(22) PCT Filed: Oct. 13, 2006
(86) PCT No.: PCT/EP2006/067401

§ 371 Date: Apr. 17, 2008

(30) Foreign Application Priority Data
Nov. 17, 2005 (EP) 05110842.1

(51) Int. Cl. C09K 3/00; C07D 401/04; C07D 251/14

(52) U.S. Cl. 252/182.29; 544/212; 544/194

(57) ABSTRACT

The invention relates to triazine compounds comprising both amino- and carboxyl-containing substituents and also to a process for preparing them.

The invention relates to triazine compounds comprising both amino- and carboxyl-containing substituents, and also to a process for preparing them.

EP 0 466 647 describes a process for the photochemical and thermal stabilization of polyamide fiber materials and also water-soluble triazine derivatives conforming to the formula I.

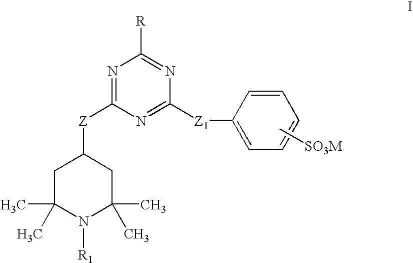

DE 195 31 995 likewise describes a process for improving the thermal and/or photochemical stability of dyed and undyed polyamide fibers. Stabilization is achieved in this process by treatment, from an aqueous bath, with an agent which comprises a compound of the formula II:

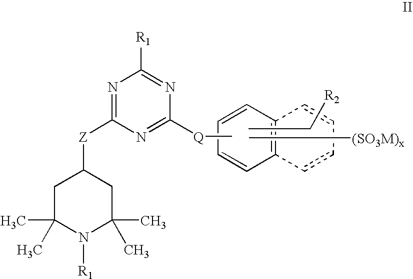

and a UV absorber.

EP 0 702 011 describes a process for the photochemical and thermal stabilization of polyamide fiber materials and dyeings. The process utilizes water-soluble piperidine-triazine compounds of the formula III

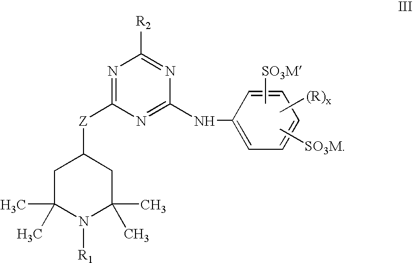

Similar compounds for the photochemical and thermal stabilization of polyamide fiber materials are also described by EP 0 546 993. Compounds conforming to the formula IV

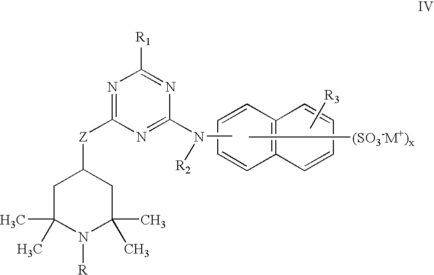

are utilized.

U.S. Pat. No. 4,883,860 describes polymeric compositions containing an effective amount of a triazine-based compound which comprises a 2,2,6,6-tetraalkylpiperidine group, as for example of the formula V.

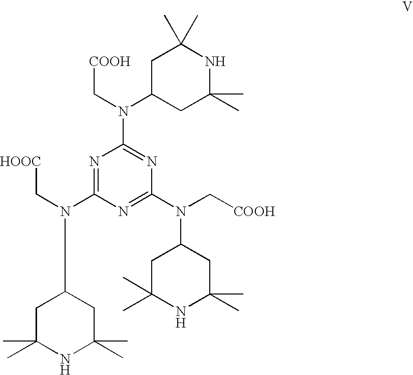

The present invention has for its object to provide compounds which are useful as permanent stabilizers for polyesters and polyamides. More particularly, these compounds shall be preparable in an economical manner, from commercially available raw materials by few reaction steps.

We have found that this object is achieved, surprisingly, by triazine compounds comprising both amino- and carboxyl-containing substituents and that they have been found to be useful as reactive stabilizers for polyesters and polyamides. Reactive stabilizers for the purposes of this invention are capable of becoming chemically bonded to the polymer, through their amino- and carboxyl-containing substituents, and hence are a building block in the polymer chain. This has the advantage over prior art stabilizers that the stabilizers of the present invention can be added at the polymerization stage and become incorporated in the polymer chain in the course of the polymerization. There is thus no need for an additional step of admixing the stabilizer to the polymer. These stabilizers of the present invention further have the advantage that they cannot be dissolved out of the polymer and thus a permanent stabilizer is available. The achievement of the object was all the more surprising since it has been determined that these compounds can be prepared by an economical process.

The present invention provides triazine compounds of the formula (1)

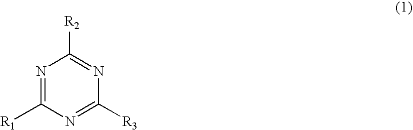

where
   R₁=-A-B
      where
         A=—O— or —NR₄—,
         B=amino-containing substituent and
         R₄=hydrogen or alkyl
   R₂=

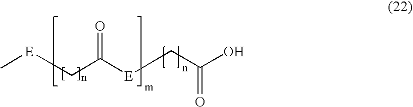

where
   E=—O— or —NR₅—,
   n=3 to 15,
   m=0 to 10 and
   R₅=hydrogen or alkyl
R₃=R₁, R₂, —OR₆ or —NR₇R₈
   where R₆, R₇ and R₈=hydrogen, alkyl or aryl, each substituted or unsubstituted.

This invention further provides a process for preparing triazine compounds conforming to the formula (1), the process being characterized in that cyanuric chloride is reacted with 0.5 to 5 mole equivalents of an amine of the formula (2)

H-A-B (2)

in the presence of a base and of 0.5 to 5 mole equivalents of a compound conforming to the formula (3)

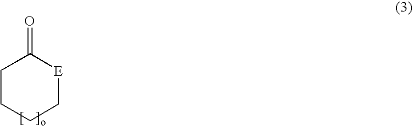

or of a compound conforming to the formula (4)

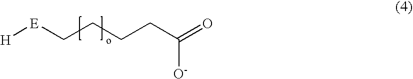

where
   o is from 0 to 12,
   E=—O— or —NR₅— and
   R₅=hydrogen or alkyl,
      and the two reaction steps may be carried out in either order.

This invention further provides a composition characterized in that the composition comprises at least two different triazine compounds conforming to the formula (1).

This invention further provides a solution characterized in that the solution comprises at least one triazine compound conforming to the formula (1).

The triazine compounds of the present invention have a structure which conforms to the formula (1)

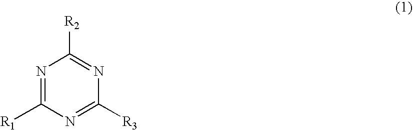

where
   R₁=-A-B
      where
         A=—O— or —NR₄—,
         B=amino-containing substituent and
         R₄=hydrogen or alkyl
   R₂=

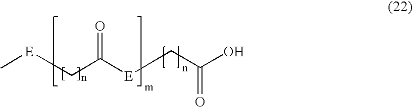

where
   E=—O— or —NR₅—,
   n=3 to 15,
   m=0 to 10 and
   R₅=hydrogen or alkyl
R₃=R₁, R₂, —OR₆ or —NR₇R₈
   where R₆, R₇ and R₈=hydrogen, alkyl or aryl, each substituted or unsubstituted.

The structural fragment A in the present invention's triazine compounds' R₁ substituent may be not only —O— but also —NR₄—, and the structural fragment A in the R₁ and R₃ substituents may be the same or different. Preferably, the structural fragment A is —NR₄—. The R₄ substituent may be not only hydrogen but also an alkyl group. Preferably, the R₄ substituent is hydrogen or an alkyl group having from 1 to 10 and preferably from 2 to 5 carbon atoms. This alkyl group of the structural fragment A may be branched or unbranched; preferably, it is unbranched. Furthermore, this alkyl group is preferably unsubstituted. More preferably, the R₄ substituent is hydrogen, however.

The B substituent is in particular an amino-containing substituent in which the amino group may be situated on an aliphatic supporting scaffold or may be an aliphatic cyclic amine. Preferably, the B substituent comprises an aliphatic cyclic amine.

Preferably, the triazine compounds of the present invention comprise a B substituent which conforms to the formula (5)

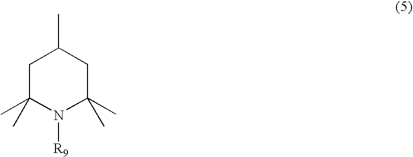

where
   R₉=hydrogen, alkyl or alkoxy of the formula —O—R₂₀,
   R₂₀=a branched or unbranched alkyl or cycloalkyl group having in either case from 4 to 16 carbon atoms,
      or which conforms to the formula (6)

—(CH₂)ₚ—NR₁₀OR₁₁ (6)
where p is from 1 to 15, preferably from 2 to 8 and more preferably from 3 to 6 and R₁₀, R₁₁=hydrogen, alkyl, cycloalkyl or heterocycloalkyl, and R₁₀ and R₁₁ are the same or different,
   in the R₁ substituent.

The R₁₀ and R₁₁ substituents may be the same or different and are preferably hydrogen, an alkyl, cycloalkyl or heterocycloalkyl group, in particular having in each case from 1 to 20 and preferably from 2 to 10 carbon atoms or from 1 to 20 and preferably from 2 to 10 carbon and hetero atoms. This alkyl group of the R₁₀ and R₁₁ substituents are preferably branched or unbranched, but more preferably they are unbranched. Furthermore, they are preferably unsubstituted or substituted by an amino group, but more preferably it is unsubstituted. The cycloalkyl group of the R₁₀ and R₁₁ substituents is preferably unsubstituted or substituted, in particular this cycloalkyl group is unsubstituted. The heterocycloalkyl group of the R₁₀ and R₁₁ substituents is preferably unsubstituted or substituted, preferably this heterocycloalkyl group is substituted by one or more methyl groups, preferably it is a heterocycloalkyl group which comprises one or more nitrogen atoms as a hetero atom, preferably it is a heterocycloalkyl group which conforms to the formula (5).

The R₉ substituent is preferably hydrogen or alkyl having from 1 to 16 and preferably from 1 to 8 carbon atoms or an alkoxy group having a branched or unbranched alkyl group or a cycloalkyl group; more preferably, the R₉ substituent is hydrogen.

Preferably, the triazine compounds of the present invention comprise a B substituent which conforms to the formula (6). More preferably, the triazine compounds of the present invention comprise a B substituent which conforms to the formula (6a) or (6b):

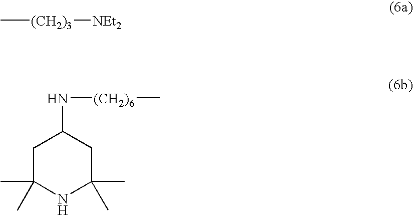

In a particularly preferred embodiment of the triazine compounds of the present invention, these comprise a B substituent which conforms to the formula (5).

Very particular preference is given to triazine compounds comprising an R₁ substituent conforming to the formula (7):

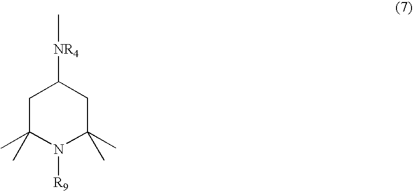

The R₂ substituents in the triazine compounds of the present invention preferably comprise a structural fragment E with —O— or —NR₅—, the R₅ substituent preferably being hydrogen or an alkyl group having from 1 to 16 and preferably from 1 to 4 carbon atoms; preferably, the R₅ substituent is hydrogen. The alkyl group of the R₅ substituent is branched or unbranched; preferably, it is unbranched. This alkyl group of the R₅ substituent is also preferably unsubstituted. The R₂ substituent has an n which is preferably in the range from 3 to 15, more preferably in the range from 5 to 11 and even more preferably 5 and an m which is preferably in the range from 0 to 10, more preferably in the range from 0 to 4 and even more preferably equal to 0.

The triazine compounds of the present invention preferably comprise an R₁ substituent as R₃ substituent. In a particular embodiment, the triazine compounds of the present invention comprise an R₂ substituent as R₃ substituent. The two R₁ and R₂ substituents may be respectively identical or different; preferably, the substituents of the same type are identical.

In a preferred embodiment, the triazine compounds of the present invention comprise an R₁ substituent which conforms to the following structures (7a), (7b) or (7c):

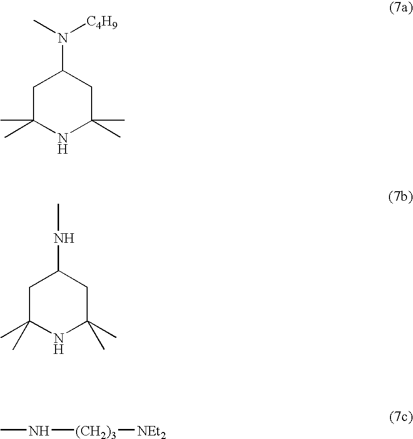

The R₂ substituents in this preferred embodiment have in particular structures conforming to the formulae (8a) or (8b):

—NH—(CH₂)₅—COOH (8a)

—NH—(CH₂)₁₁—COOH (8b)

In this preferred embodiment, the R₃ substituent is preferably an R₁ substituent and more preferably these two substituents are identical. In this preferred embodiment the R₃ substituent may also be an R₂ substituent and again these two substituents are preferably identical.

In a further embodiment of the triazine compounds of the present invention, these comprise an R₃ substituent which conforms to the following formulae (14) and (15):

—O—R₆ (14)

or

—NR₇R₈, (15)

where R₆, R₇ and R₈ are each hydrogen, alkyl or aryl, the alkyl or aryl group being unsubstituted or substituted by one or more substituents of the formula R₆, R₇, R₈, —SO₃H or —SO₃M, where M is an alkali metal cation and preferably the alkali metal cation is a lithium, sodium or potassium cation. The alkyl and/or aryl groups of the R₆, R₇ or R₈ substituents preferably comprise from 1 to 4 substituents, more preferably from 1 to 2 substituents and most preferably one substituent of the formula —SO₃H or —SO₃M. Preferably, the R₆, R₇ and R₈ substituents are hydrogen or an alkyl group having from 1 to 18 and preferably from 2 to 16 carbon atoms. This alkyl group of the R₆, R₇ and R₈ substituent may be branched or unbranched; preferably, it is unbranched. The aryl group of the R₆, R₇ and R₈ substituents is preferably an unsubstituted phenyl group or a phenyl group which is monosubstituted, preferably in the para position, by —SO₃H or —SO₃M. The R₆, R₇ and R₈ substituents may be all identical, all different or else form identical pairs. Preferably, the R₆, R₇ and R₈ substituents are hydrogen, unsubstituted phenyl or phenyl which is monosubstituted by —SO₃H or —SO₃M in the para position.

The present invention's process for preparing triazine compounds conforming to the formula (1),

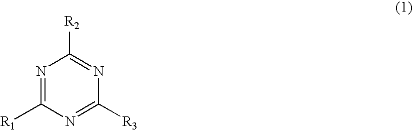

where
   R₁=-A-B
      where
         A=—O— or —NR₄—,
         B=amino-containing substituent and
         R₄=hydrogen or alkyl
   R₂=

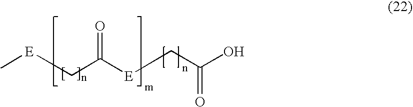

where
   E=—O— or —NR₅—,
   n=3 to 15,
   m=0 to 10 and
   R₅=hydrogen or alkyl
R₃=R₁, R₂, —OR₆ or —NR₇R₈
   where R₆, R₇ and R₈=hydrogen, alkyl or aryl, each substituted or unsubstituted,
      is characterized in that cyanuric chloride is reacted with 0.5 to 5 mole equivalents of an amine of the formula (2)

H-A-B (2)

in the presence of a base and of 0.5 to 5 mole equivalents of a compound conforming to the formula (3)

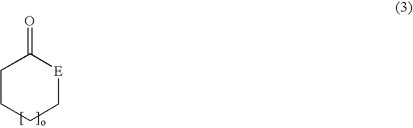

or of a compound conforming to the formula (4)

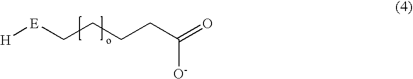

where
   o is from 0 to 12,
   E=—O— or —NR₅— and
   R₅=hydrogen or alkyl,
      and the two reaction steps may be carried out in either order.

That embodiment of the present invention's process wherein compounds having R₃ other than R₁ or R₂ are prepared utilizes a further reaction step wherein 0.5 to 5 mole equivalents of a compound conforming to the formula (16) or (17)

H—OR₆ (16)

or

H—NR₇R₈ (17)

are reacted in the presence of a base.

The R₅ substituent may be not only hydrogen but also an alkyl group. Preferably, the R₅ substituent is hydrogen or an alkyl group having from 1 to 16 and preferably from 1 to 4 carbon atoms. This alkyl group of the R₅ substituent may be branched or unbranched, preferably unbranched. Furthermore, this alkyl group of the R₅ substituent is preferably unsubstituted. Preferably, however, R₅ is hydrogen. In the compounds conforming to the formulae (3) or (4), o is preferably in the range from 0 to 12, more preferably in the range from 2 to 8 and even more preferably 2.

The process of the present invention preferably utilizes lactams or lactones and more preferably lactams as compounds conforming to the formula (3). It is particularly preferable for caprolactam to be used in the process of the present invention. One particular embodiment of the process of the present invention utilizes a compound which conforms to the formula (4), more preferably sodium aminocaproate.

The reactant conforming to the formula (4) may also be formed in situ in the process of the present invention, for example through the use of a compound conforming to the formula (3) and a base, in particular an alkali metal hydroxide, such as sodium hydroxide for example. The amount of substance ratio in which the two reactants are used for this is preferably in the range from 5:1 to 1:1, more preferably in the range from 4:1 to 1:1 and even more preferably in the range from 2:1 to 1:1.

The process of the present invention utilizes from 0.5 to 5, preferably from 1 to 3 and more preferably from 1 to 2 mole equivalents of the compound conforming to the formula (3) or (4) based on the cyanuric chloride used.

The process of the present invention preferably utilizes as a further reactant an amine conforming to the formula (2), and the structural fragment A may be not only —O— but also —NR₄—. Preference is given to using an amine which conforms to the formula (2) and comprises —NR₄— as structural fragment A. The R₄ substituent may be not only hydrogen but also an alkyl group. Preferably the R₄ substituent is hydrogen or an alkyl group having from 1 to 10 and preferably from 2 to 5 carbon atoms. This alkyl group of the structural fragment A may be branched or unbranched, preferably it is unbranched. Furthermore, this alkyl group is preferably unsubstituted. More preferably, however, the R₄ substituent is hydrogen.

The process of the present invention utilizes an amine of the formula (2) where the B substituent is preferably an amino-containing substituent in which the amino group may be situated on an aliphatic supporting scaffold or may be an aliphatic cyclic amine. Preferably, this B substituent comprises an aliphatic cyclic amine.

The process of the present invention preferably utilizes amines of the formula (2) which comprise a B substituent of the formula (6) or of the formula (5).

The R₁₀ and R₁₁ substituents may be the same or different and are preferably hydrogen, an alkyl, cycloalkyl or heterocycloalkyl group, having in each case from 1 to 20 and preferably from 2 to 10 carbon atoms or from 1 to 20 and preferably from 2 to 10 carbon and hetero atoms. This alkyl group of the R₁₀ and R₁₁ substituents are preferably branched or unbranched, but more preferably they are unbranched. Furthermore, they are preferably unsubstituted or substituted by an amino group, but more preferably it is unsubstituted. The cycloalkyl group of the R₁₀ and R₁₁ substituents is preferably unsubstituted or substituted, in particular this cycloalkyl group is unsubstituted. The heterocycloalkyl group of the R₁₀ and R₁₁ substituents is preferably unsubstituted or substituted, preferably this heterocycloalkyl group is substituted by one or more methyl groups, preferably it is a heterocycloalkyl group which comprises one or more nitrogen atoms as a hetero atom, preferably it is a heterocycloalkyl group which conforms to the formula (5).

The R₉ substituent is preferably hydrogen or alkyl having from 1 to 16 and preferably from 1 to 8 carbon atoms or an alkoxy group comprising a branched or unbranched alkyl group or a cycloalkyl group, more preferably, the R₉ substituent is hydrogen.

The process of the present invention preferably utilizes amines having a B substituent which conforms to the formula (6). However, it is particularly preferable to utilize amines having a B substituent which conforms to the formula (6a) or (6b).

Very particular preference is given to utilizing amines conforming to the formula (2a)

HNR₄—(CH₂)ₘ—NR₁₀R₁₁ (2a)

One particularly preferred embodiment of the process according to the present invention utilizes amines comprising a B substituent conforming to the formula (5). It is very particularly preferable to utilize amines conforming to the formula (2b):

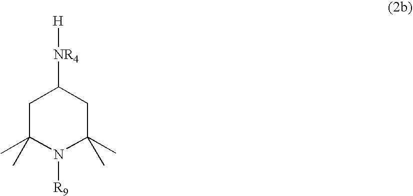

in the process of the present invention.

The process of the present invention may also utilize mixtures of different compounds conforming to the formulae (3) or (4) or else mixtures of different amines conforming to the formula (2).

A further embodiment of the process according to the present invention utilizes, as a further reactant, compounds conforming to the formula (16) or (17), where R₆, R₇ and R₈ are each hydrogen, alkyl or aryl, the alkyl or aryl group being unsubstituted or substituted by one or more substituents of the formula R₆, R₇, R₈, —SO₃H or —SO₃M, where M is an alkali metal cation and preferably the alkali metal cation is a lithium, sodium or potassium cation. The alkyl and/or aryl groups of the R₆, R₇ or R₈ substituents preferably comprise from 1 to 4 substituents of the formula —SO₃H or —SO₃M, more preferably comprise from 1 to 2 substituents of the formula —SO₃H or —SO₃M and most preferably comprise one substituent of the formula —SO₃H or —SO₃M. Preferably, the R₆, R₇ and R₈ substituents are hydrogen or an alkyl group having from 1 to 18 and preferably from 2 to 16 carbon atoms. This alkyl group of the R₆, R₇ and R₈ substituent may be branched or unbranched; preferably, it is unbranched. The aryl group of the R₆, R₇ and R₈ substituents is preferably an unsubstituted phenyl group or a phenyl group which is monosubstituted, preferably in the para position, by —SO₃H or —SO₃M. The R₆, R₇ and R₈ substituents may be all identical, all different or else form identical pairs. Preferably, the R₆, R₇ and R₈ substituents are hydrogen, unsubstituted phenyl or phenyl which is monosubstituted by —SO₃H or —SO₃M in the para position.

Depending on the triazine compound to be prepared, the process of the present invention may consist of two or three reaction steps for the actual conversion or reaction.

One preferred embodiment of the process of the present invention comprises reacting cyanuric chloride with an amine conforming to the formula (2) in the presence of a base in a solvent in a first reaction step. Aqueous sodium hydroxide solution is preferably used as base. Amine and base are preferably used in an amount of substance ratio of 1:1. This first reaction step of this preferred embodiment may utilize a solvent selected from water, aromatic hydrocarbons, in particular toluene, xylene, alkanes, ethers, ketones, such as acetone for example, or esters; water is preferably used as solvent. Alcohols, primary or secondary amines are unsuitable as solvents for this first reaction step. The second reaction step then comprises the reaction with a compound selected from compounds conforming to the formula (3) or (4).

A further embodiment of the process of the present invention comprises a first reaction step in which cyanuric chloride is reacted with a compound selected from compounds conforming to the formula (3) or (4) and subsequently, in a further reaction step, with an amine conforming to the formula (2). The solvents and the amount of substance ratio of amine to base may be chosen similarly to the preferred embodiment.

The reaction step involving a compound conforming to the formula (3) as a reactant is preferably carried out in the presence of a ring-opening base. In particular water, toluene, xylene, alkanes, ethers, ketones, such as acetone for example, or esters, but preferably water are used here as solvent. One particular embodiment of the process of the present invention utilizes a lactam solvent, more particularly a compound conforming to the formula (3), and it is more preferable to utilize the same compound as solvent and as reactant. However, when a compound conforming to the formula (4) is utilized as a reactant in a reaction step, the reaction step is carried out in the presence of an excess of the corresponding compound conforming to the formula (3), meaning an amount of substance ratio of compound conforming to the formula (3) to cyanuric chloride which is preferably 1:4 and in particular 1.1:3.5. For example, when sodium aminocaproate is used as a formula (4) compound reactant, the reaction is carried out in the presence of an excess of caprolactam. When the process of the present invention is used for preparing triazine compounds conforming to the formula (1) where R₃=R₁ or R₂, the process of the present invention comprises a first reaction step in which the cyanuric chloride is reacted with from 0.5 to 3 and preferably from 1 to 2 mole equivalents based on the amount of substance of cyanuric chloride of reactant A, reactant A being either an amine conforming to the formula (2) or a compound selected from compounds conforming to the formula (3) or (4). In a second reaction step of the process according to the present invention, the intermediate obtained is then reacted with from 0.5 to 5 and preferably from 1 to 4 mole equivalents based on the amount of substance of cyanuric chloride of reactant B, reactant B being
a compound selected from the compounds conforming to the formula (3) or (4) when an amine conforming to the formula (2) is used as reactant A, or
an amine conforming to the formula (2) when a compound selected from compounds conforming to the formula (3) or (4) is used as reactant A.

The temperature at which the first reaction step of the process of the present invention is carried out is preferably in the range from −20 to 100° C., more preferably in the range from −10 to 80° C. and even more preferably in the range from 0 to 60° C. By contrast, the temperature at which the second reaction step is carried out is preferably in the range from 0 to 200° C., more preferably in the range from 10 to 180° C. and even more preferably in the range from 20 to 170° C. In the case of an embodiment of the process of the present invention where only one chlorine atom of the cyanuric chloride is reacted with reactant A in the first reaction step and the two remaining chlorine atoms of the cyanuric chloride are reacted with reactant B in a second reaction step, it is advantageous to employ a temperature ramp in the second reaction step.

The pressure at which the first reaction step of the process of the present invention is carried out is preferably in the range from 0.5 to 1.5 bar, more preferably in the range from 0.8 to 1.2 bar and even more preferably atmospheric pressure. By contrast, the pressure at which the second reaction step is carried out is preferably in the range from 1 to 11 bar, more preferably in the range from 1 to 9 bar and even more preferably in the range from 1 to 8 bar.

A further embodiment of the process of the present invention comprises a first reaction step in which cyanuric chloride is reacted with a compound selected from compounds conforming to the formula (3) or (4) in a solvent. The reaction with an amine conforming to the formula (2) is then carried out in the second reaction step.

In one preferred embodiment of the process of the present invention, the first reaction step comprises reacting the cyanuric chloride with from 1 to 3 mole equivalents and preferably with 2 mole equivalents based on the amount of substance of cyanuric chloride of reactant A and a second reaction step which then comprises reacting the resulting intermediate with 0.5 to 5 and preferably from 1 to 3 mole equivalents based on the amount of substance of cyanuric chloride of reactant B. The temperature at which the first reaction step is carried out is preferably in the range from 0 to 100° C., more preferably in the range from 10 to 80° C. and even more preferably in the range from 20 to 60° C. By contrast, the temperature at which the second reaction step is carried out is preferably in the range from 80 to 200° C., more preferably in the range from 90 to 180° C. and even more preferably in the range from 100 to 170° C.

When the process of the present invention is used for preparing triazine compounds conforming to the formula (1) where R₃=—OR₆ or —NR₇R₈, then the process of the present invention comprises a first reaction step where cyanuric chloride is reacted with from 0.5 to 2 mole equivalents and preferably 1 mole equivalent based on the amount of substance of cyanuric chloride of reactant A, reactant A being either an amine conforming to the formula (2) or a compound selected from compounds conforming to the formula (3) or (4), or a hydroxy or amino compound of the formulae (16) or (17). In a second reaction step of the process of the present invention, the intermediate obtained is then reacted with from 0.5 to 2 mole equivalents and preferably with 1 mole equivalent based on the amount of substance of cyanuric chloride of reactant B, reactant B being
a compound selected from compounds conforming to the formulae (3), (4), (16) or (17) when a compound of the formula (2) is used as reactant A, or
a compound selected from compounds conforming to the formula (2), (16) or (17) when a compound selected from the compounds conforming to the formula (3) or (4) is used as reactant A, or
a compound selected from compounds conforming to the formula (2), (3) or (4) when a compound selected from compounds conforming to the formula (16) or (17) is used as reactant A.

In a third reaction step of this particular embodiment of the process of the present invention, the intermediate obtained is then reacted with from 0.5 to 2 mole equivalents and preferably with 1 mole equivalent based on the amount of substance of cyanuric chloride of reactant C, reactant C being
a compound selected from compounds conforming to the formulae (16) or (17) when compounds of the formula (2) and (3) or (4) are used as reactants A and B, or
a compound selected from compounds conforming to the formulae (3) or (4) when compounds of the formulae (3) or (4) and (16) or (17) are used as reactants A and B, or
a compound conforming to the formula (2) when compounds of the formula (3) or (4) and (16) or (17) are used as reactants A and B.

The temperature at which the first reaction step of this particular embodiment of the process of the present invention is carried out is preferably in the range from −20 to 80° C., more preferably in the range from −10 to 60° C. and even more preferably in the range from 0 to 40° C. The temperature at which the second reaction step is carried out is by contrast preferably in the range from 0 to 100° C., more preferably in the range from 10 to 80° C. and even more preferably in the range from 20 to 60° C. The temperature at which the third reaction step is carried out is preferably in the range from 80 to 200° C., more preferably in the range from 90 to 180° C. and even more preferably in the range from 100 to 170° C.

The pressure at which the first and second reaction steps of this particular embodiment of the process of the present invention are carried out is preferably in the range from 0.5 to 1.5 bar, more preferably in the range from 0.8 to 1.2 bar and even more preferably equal to atmospheric pressure. The pressure at which the third reaction step is carried out is by contrast preferably in the range from 1 to 11 bar, more preferably in the range from 1 to 9 bar and even more preferably in the range from 1 to 8 bar.

The individual reaction steps of the process of the present invention can be carried out in any one stage of the process of the present invention, in which case the intermediates which are formed in each case may be separated off and isolated and thus may be used as a reactant for the next stage.

In one particular embodiment, the intermediates of any one stage are not separated off and isolated—with the exception of the last stage—but are directly fed as a reactant to the next stage. In this embodiment of the process of the present invention, the intermediates are thus formed in situ.

In another embodiment of the process of the present invention, all reaction steps are carried out in one reaction apparatus, in particular in an autoclave. In this embodiment, the reaction of the compounds conforming to the formula (3) with a base can be carried out in a separate stage or reaction vessel. However, the reaction of the compounds conforming to the formula (3) with a base can also be carried out in the same reaction vessel, so that all reaction steps of the process of the present invention take place in the same reaction vessel. In this way, all three substitutions on the triazine ring of the process of the present invention can be carried out in one reaction vessel.

In general, the reaction of the compounds conforming to the formula (3) with a base can take place in a separate stage or reaction vessel. However, the reaction of the compounds conforming to the formula (3) with a base can also take place in the same reaction vessel in which the respective reaction stage is just taking place.

The work-up of the reaction mixture chiefly serves to remove the byproduced sodium chloride. When the triazine compounds of the present invention are insoluble in water, the sodium chloride is dissolved in water and removed by filtering the aqueous suspensions and subsequently washing the filter cake or by extracting the target product with an organic solvent, preferably with the organic solvent employed during the reaction. When the triazine compounds are soluble in water, the likewise water-soluble sodium chloride is preferably removed by electrodialysis via membranes or by means of ion exchange chromatography; the sodium chloride is preferably removed by ion exchange chromatography.

The reaction mixture thus worked up, which constitutes a solution in water or in an organic solvent, can then be used directly; there is one particular embodiment where the use as a stabilizer is preceded by a drying operation.

The intermediates arising after the individual reaction steps may in one particular embodiment of the process of the present invention be isolated from the reaction mixture and purified. This is preferably accomplished by crystallization, filtration and if appropriate a wash from the reaction mixture. The isolating and purifying of these intermediates can also be effected by means of an extraction with an organic solvent, preferably with the organic solvent already employed during the reaction. The intermediates thus isolated and purified are generally solids and can then be employed in the next reaction step of the process of the present invention.

In one preferred embodiment of the process of the present invention, however, these intermediates are not isolated and worked up. The reaction steps of the process are carried out in succession without the intermediates being isolated and worked up.

The compositions of the present invention comprise at least two different triazine compounds conforming to the formula (1).

The composition of the present invention preferably comprises
85% to 95% by weight of present invention triazine compounds conforming to the formula (18),

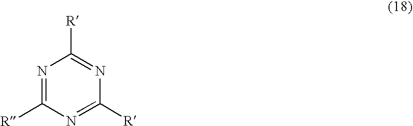

0% to 10% by weight of triazine compounds conforming to the formula (19),

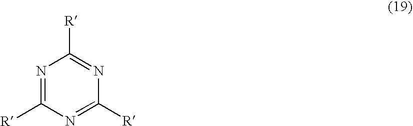

0% to 10% by weight of triazine compounds conforming to the formula (20),

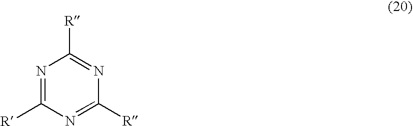

where R′=R₁ or R₂ and R″=R₂ or R₁ and R′ is not identical to R″, meaning in particular that when R′=R₁, then R″=R₂ and vice versa.

In one preferred embodiment of the composition of the present invention R′=R₁ and R″=R₂; this composition is obtainable when the reactants cyanuric chloride, amine of formula (2) and a compound conforming to formula (3) or (4) are used in an amount of substance ratio of 1:2:1 in the process of the present invention.

In one particularly preferred embodiment of the composition of the present invention R′=R₂ and R″=R₁; this composition is obtainable when the reactants cyanuric chloride, amine of formula (2) and a compound conforming to formula (3) or (4) are used in an amount of substance ratio of 1:1:2 in the process of the present invention.

A further embodiment of the composition of the present invention comprises
30% to 95% by weight of present invention triazine compounds conforming to the formula (18),

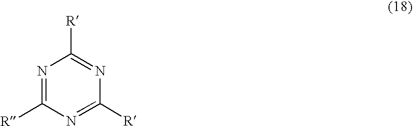

0% to 60% by weight of present invention triazine compounds conforming to the formula (20),

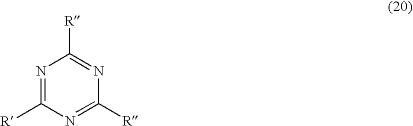

0% to 10% by weight of triazine compounds conforming to the formula (19) and (21),

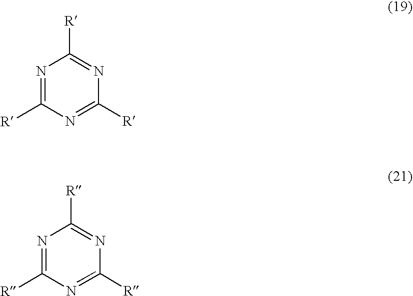

where R′=R₁ or R₂ and R″=R₂ or R₁ and R′ is not identical to R″, meaning in particular that when R′=R₁, then R″=R₂ and vice versa. This composition is obtainable when the reactants cyanuric chloride, amine of formula (2) and a compound conforming to formula (3) or (4) are used in an amount of substance ratio of 1:1.5:1.5 in the process of the present invention.

A further preferred embodiment of the composition of the present invention comprises
60% to 90% by weight of present invention triazine compounds conforming to the formula (23),

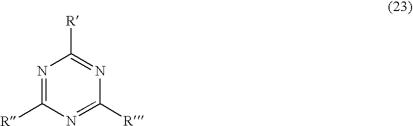

1% to 30% by weight of present invention triazine compounds each having two substituents of the same type and a different third substituent selected from R′, R″ or R′″, for example triazine compounds conforming to the formula (18) or (20),
0.5% to 10% by weight of triazine compounds where all three substituents are of the same type selected from R′, R″ or R′″, for example triazine compounds conforming to the formula (19) or (21),
   where R′=R₁, R₂ or R₃, R″=R₂, R₁ or R₃ and R′″=R₃, R₂ or R₁ and R′, R″ and R′″ are not identical, meaning in particular that when R′=R₁, then R″=R₂ and R′″=R₃, or vice versa. This composition is obtainable when the reactants cyanuric chloride, amine of the formula (2), a compound conforming to the formula (3) or (4) and a compound conforming to the formula (16) or (17) are used in an amount of substance ratio of 1:(from 0.5 to 2):(from 0.5 to 2):(from 0.5 to 5) and preferably in an amount of substance ratio of 1:1:1:(from 1 to 4) in the process of the present invention.

The solution of the present invention comprises at least one triazine compound conforming to the formula (1) and preferably it comprises from 1% to 50% by weight and more preferably from 20% to 45% by weight of triazine compounds conforming to the formula (1). The solution of the present invention preferably comprises as solvent water, an organic solvent selected from aromatic hydrocarbons, in particular toluene, xylene, alkanes, ethers, ketones, for example acetone, or esters, or the compound conforming to the formula (3) which is used for preparing the triazine compounds of the present invention. Preferably, the solution of the present invention comprises water as solvent.

The present invention's use of the triazine compounds conforming to the formula (1) is for stabilizing polymers.

The triazine compounds of the present invention can be used for stabilizing polyamides in particular. Possible polyamides include primarily aliphatic homo- and copolycondensates, examples being PA 46, PA 66, PA 68, PA 610, PA 612, PA 410, PA 810, PA 1010, PA 412, PA 1012, PA 1212, PA 6, PA 7, PA 8, PA 9, PA 10, PA 11 and PA 12. The triazine compounds of the present invention are preferably used for stabilizing PA 410, PA 810, PA 1010, PA 412, PA 1012, PA 1212, PA 6, PA 7, PA 8, PA 9, PA 10, PA 11 and PA 12. This designation of the polyamides conforms to international practice where the first digit or digits indicates the number of carbon atoms of the starting diamine and the last digit or digits indicate or indicates the number of carbon atoms of the dicarboxylic acid. Where there is only one number, the respective polyamide was prepared from an α,ω-aminocarboxylic acid or from the lactam derived therefrom. Also see H. Domininghaus, “Die Kunststoffe and ihre Eigenschaften”, pages 272 ff., VDI-Verlag, 1976.

The triazine compounds of the present invention can further be used for stabilizing polyester. More particularly, they can be used for stabilizing polyesters prepared by polycondensation of diols with dicarboxylic acid or their polyester-forming derivatives such as dimethyl esters. Suitable diols have the formula HO—R—OH, where R is a divalent, branched or unbranched aliphatic and/or cycloaliphatic radical having 2 to 18 and preferably 2 to 12 carbon atoms. Suitable dicarboxylic acids have the formula HOOC—R′—COOH, where R′ is a divalent aliphatic, cycloaliphatic or aromatic radical having 2 to 18 and preferably 4 to 12 carbon atoms. The preparation of these polyesters is prior art (German Offenlegungsschrifts 24 07 155, 24 07 156; Ullmanns Encyclopadie der technischen Chemie, 4th edition, volume 19, pages 65 ff., Verlag Chemie, Weinheim, 1980).

The triazine compounds of the present invention can be added as early as the polycondensation stage; this has the advantage that the polyamides thus prepared will comprise the additives, which improve the mechanical strength, the stability against oxidative and light-induced degradation and the dyeability, in covalent and hence permanent attachment. The triazine compounds of the present invention are added to the polycondensation such that the triazine compounds of the present invention can become incorporated into the polymer chain by means of their functional groups.

The examples which follow illustrate the process of the present invention and also the triazine compounds of the present invention without limiting the invention to this embodiment.

### EXAMPLE 1

### Preparation of N-[4,6-bis(N-[2,2,6,6-tetramethyl-4-piperidylamino])-1,3,5-triazin-2-yl]-6-aminohexanoic acid

### 1.1 Preparation of the Intermediate 2-chloro-4,6-bis-(N-[2,2,6,6-tetramethyl-4-piperidyl-amino])-1,3,5-triazine

A 6 l glass reaction flask was charged with 276.6 g of cyanuric chloride (1.5 mol) in 3 l of water at 5° C. After 468.9 g of 4-amino-2,2,6,6-tetramethylpiperidine (3.0 mol) have been added at 5 to 20° C. and 480 g of 25% by weight aqueous sodium hydroxide solution (corresponding to 3.0 mol of sodium hydroxide) at 20 to 25° C., the mixture was stirred one hour at 25° C. and one hour at 60° C. After cooling to 25° C., the solid obtained was filtered off and washed three times with 2 l of water each time and dried at 15 mbar and 100° C. for 20 hours. The yield was 597.0 g (1.41 mol, 94% based on amount of cyanuric chloride used).

### 1.2 Preparation of Crude Product

In a 2 l glass reaction flask, 565.8 g of ε-caprolactam (5.0 mol) were melted at 80° C. After 160 g of 25% by weight aqueous sodium hydroxide solution (corresponding to 1.0 mol of sodium hydroxide), the mixture was heated at 126 to 130° C. for 2 hours with stirring. This molten solution of sodium aminocaproate in ε-caprolactam was admixed, at 110° C., with 424.0 g of the intermediate prepared as described in Example 1.1 (1 mol). This suspension was refluxed for 3 hours at 126 to 135° C. under atmospheric pressure. After cooling to 80° C., 800 ml of ethyl acetate were added to crystallize the crude product. After cooling to 40° C., the solid was filtered off, washed twice with 400 ml of ethyl acetate each time to remove excess ε-caprolactam and dried at 15 mbar and 50° C. The yield was 545.2 g.

### 1.3 Work-Up of Crude Product

### a.) Using Electrodialysis:

60.8 g of the crude product (from Example 1.2) were dissolved in 547 g of water. The resulting solution, about 10% by weight in strength, was desalted in an electrodialysis apparatus (3 circuit stacked construction/platinum anode, VA steel cathode/AHA 1 anode exchanger membrane, C 22 cathode exchanger membrane/anode-cathode circuit solution: 5% aqueous sodium sulfate solution, salt receiver circuit solution: 3% aqueous sodium chloride solution/voltage 6 V) for 24 hours at 25° C. and a current strength from 1.96 to 0.0 A. The product solution was evaporated at 130° C. and atmospheric pressure and the solid was thereafter dried at 90° C. and 15 mbar. The yield was 54.2 g (0.10 mol, 86% based on utilized amount of intermediate from 1.1).

### P Analysis of Product by HPLC (CLND):

| Composition of product | [in % by weight] |
|---|---|
| Inventive triazine compound | 92.1 |
| (as per formula (1) with | |
| R₁ as per formula (7b) and | |
| R₂ with E = —NH— and n = 5) | |
| R₂ with m = 0; R₃ = R₁ | 83.3 |
| R₂ with m = 1-3; R₃ = R₁ | 8.1 |
| R₂ with m = 0; R₃ = —OH | 0.7 |
| ε-Caprolactam | 7.9 |

The composition of the products were determined using HPLC and a chemiluminescence nitrogen detector (CLND). This kind of detector permits the equimolar detection of nitrogenous compounds.

b) Using Ion Exchange chromatography:

A 1200 ml glass column 40 cm in height and 6.2 cm in diameter was used at 25° C. The ion exchanger resin used was Amberlyst 35 from Rohm and Haas (1.9 eq. H⁺/l, 5.2 eq. H⁺/kg). Desalted water was used as solvent. The eluents were checked by measuring the pH values and the refractive indices. 1060 ml (2.0 mol of H⁺ equivalents) of the strongly acidic ion exchanger resin were presented in the H⁺ form. Following addition of 314.4 g of crude product from Example 1.2 in the form of a 25% by weight aqueous solution, the system was rinsed with water until a pH of 6 to 7 was reached. The product was eluted with 360 ml of 14% by weight aqueous NH₄OH solution. The ion exchanger was regenerated with 10% sulfuric acid after rinsing with deionized water to pH 7-8. The entire eluate with the product was evaporated at 40° C. and 30 mbar and the solid obtained was dried at 15 mbar and 100° C. The yield was 258.7 g (0.50 mol, 87% based on utilized amount of intermediate from 1.1).

### Analysis of Product by HPLC (CLND):

| Composition of product | [in % by weight] |
|---|---|
| Inventive triazine compound | 100.0 |
| (as per formula (1) with | |
| R₁ as per formula (7b) and | |
| R₂ with E = —NH— and n = 5) | |
| R₂ with m = 0; R₃ = R₁ | 91.0 |
| R₂ with m = 1-3; R₃ = R₁ | 8.3 |
| R₂ with m = 0; R₃ = —OH | 0.7 |

### EXAMPLE 2

### Preparation of N-[4,6-bis(N-[3-(diethylamino)-1-propylamino])-1,3,5-triazin-2-yl]-6-aminohexanoic acid

### 2.1 Preparation of the Intermediate 6-chloro-2,4-bis(N-[3-(diethylamino)-1-propylamino])-1,3,5-triazine

276.6 g of cyanuric chloride (1.5 mol) were reacted in 3 l of water at 5 to 20° C. first with 390.7 g of 3-(diethylamino)-1-propylamine (3.0 mol) and then at 20° C. with 480.0 g of 25% by weight aqueous sodium hydroxide solution (corresponding to 3.0 mol of sodium hydroxide) and stirred at 40° C. for one hour. The solid was filtered off at 25° C., washed twice with 1 l of water each time and dried at 15 mbar and 80° C. for 20 hours. The yield was 457.5 g (1.23 mol, 82% based on amount of cyanuric chloride used).

### 2.2 Preparation of Crude Product

A solution, molten at 110° C., of sodium aminocaproate (1.0 mol) in ε-caprolactam (4 mol) was prepared similarly to Example 1.2. After addition of 371.9 g of the intermediate prepared as described in Example 2.1 (1.0 mol) the mixture was refluxed at 125 to 133° C. under atmospheric pressure for 3 hours. Crystallization, filtration, washing with ethyl acetate and drying similarly to Example 1.2 gives 499.1 g of the crude product.

### 2.3 Work-Up of Crude Product

Example 1.3 b.) was repeated to desalt and purify 290.1 g of the crude product from Example 2.2 via ion exchanger chromatography. The yield was 232.8 g (0.50 mol, 86% based on utilized amount of intermediate from Example 2.1).

### Analysis of Product by HPLC (CLND):

| Composition of product | [in % by weight] |
|---|---|
| Inventive triazine compound | 100.0 |
| (as per formula (1) with | |
| R₁, R₃ as per formula (7c) and | |
| R₂ with E = —NH—, n = 5) | |
| R₂ with m = 0; R₃ = R₁ | 93.8 |
| R₂ with m = 1-3; R₃ = R₁ | 5.4 |
| R₂ with m = 0; R₃ = —OH | 0.8 |

### EXAMPLE 3

### Preparation of N-[4,6-bis(N-[2,2,6,6-tetramethyl-4-piperidyl]-N-butylamino)-1,3,5-triazin-2-yl]-6-aminohexanoic acid

### 3.1 Preparation of the Intermediate 6-chloro-2,4-bis(N-[2,2,6,6-tetramethyl-4-piperidyl]-N-butylamino)-1,3,5-triazine

A 6 l glass reaction flask was charged with 276.6 g of cyanuric chloride (1.5 mol) in 3 l of water at 5° C. After 637.2 g of 4-N-butylamino-2,2,6,6-tetramethylpiperidine (3 mol) have been added at 5 to 20° C. and 480 g of 25% by weight aqueous sodium hydroxide solution (corresponding to 3.0 mol of sodium hydroxide) at 20 to 25° C., the mixture was stirred one hour at 25° C. and one hour at 60° C. After cooling to 25° C., the solid obtained was filtered off and washed three times with 2 l of water each time and dried at 15 mbar and 100° C. for 20 hours. The yield was 773.8 g (1.44 mol, 96% based on amount of cyanuric chloride used)

### 3.2 Preparation of Crude Product

A solution, molten at 110° C., of sodium aminocaproate (1.0 mol) in ε-caprolactam (4 mol) was prepared similarly to Example 1.2. After addition of 536.3 g of the intermediate prepared as described in Example 3.1 (1.0 mol) the mixture was refluxed at 128 to 136° C. under atmospheric pressure for 3 hours. Crystallization, filtration, washing with ethyl acetate and drying similarly to Example 1.2 gives 670.8 g of the crude product (91% based on utilized amount of intermediate from Example 3.1).

### 3.3 Work-Up of Crude Product

Example 1.3 b.) was repeated to desalt and purify 367.7 g of crude product via ion exchanger chromatography. The yield was 313.0 g (0.50 mol, 90% based on utilized amount of intermediate from Example 3.1).

### Analysis of Product by HPLC (CLND):

| | [in % by |
|---|---|
| Composition of product | weight] |
| Inventive triazine compound | 100.0 |
| (as per formula (1) with | |
| R₁, R₃ as per formula (7a) and | |
| R₂ with E = —NH—, n = 5) | |
| R₂ with m = 0; R₃ = R₁ | 92.4 |
| R₂ with m = 1-3; R₃ = R₁ | 7.0 |
| R₂ with m = 0; R₃ = —OH | 0.6 |

### EXAMPLE 4

### Preparation of N,N′-[6-(N-[2,2,6,6-tetramethyl-4-piperidylamino])-1,3,5-triazine-2,4-diyl]bis(6-aminohexanoic acid)

### 4.1 Preparation of the Intermediate N,N′-[6-chloro-1,3,5-triazine-2,4-diyl]bis(6-aminohexanoic acid)

In a 4 l glass reaction flask, 452.6 g of ε-caprolactam (4.0 mol) were reacted with 640 g of 25% by weight aqueous sodium hydroxide solution (corresponding to 4.0 mol of sodium hydroxide) at 120 to 126° C. for one hour. The sodium aminocaproate solution was diluted with 1500 ml of water, cooled down to 5° C. and reacted with 368.8 g of cyanuric chloride (2.0 mol) at 5 to 12° C. and stirred at 20 to 25° C. for one hour. The solid was filtered off at 25° C., washed twice with 1 l of water each time and dried at 15 mbar and 80° C. for 20 hours. The yield was 593.9 g (1.54 mol, 77% based on amount of cyanuric chloride used).

### Analysis of Intermediate by HPLC (CLND):

| Composition of intermediate | [in % by weight] |
|---|---|
| Triazine compound | 96.4 |
| (analogous to formula (1) with | |
| R₁ = Cl and | |
| R₂ with E = —NH— and n = 5) | |
| R₂ with m = 0; | 85.0 |
| R₃ = R₂, where E = —NH—, | |
| n = 5 and m = 0 | |
| R₂ with m = 1-3; | 10.0 |
| R₃ = R₂, where E = —NH—, | |
| n = 5 and m = 0-3 | |
| R₂ with m = 0-1; | 1.4 |
| R₃ = —OH | |
| Aminocaproic acid | 3.6 |

### 4.2 Preparation of Crude Product

186.9 g of the intermediate prepared as described in Example 4.1 (0.5 mol) were reacted in a 2 l glass reaction flask in 1 l of water with 78.2 g of 4-amino-2,2,6,6-tetramethylpiperidine (0.5 mol) at 25 to 40° C. and with 80.0 g of 25% by weight aqueous sodium hydroxide solution (corresponding to 0.5 mol of sodium hydroxide) at 35 to 40° C. and heated at 102 to 104° C. for 3 hours. 1345 g of filtrate were obtained after cooling and depressurizing.

### 4.3 Work-Up of Crude Product

The filtrate was desalted and purified through 1000 ml of Amberlyst 35 (1.9 mol H⁺) similarly to Example 1.3.b). The yield was 235.8 g (0.48 mol, 96% based on amount of intermediate 4.1 used).

### Analysis of Product by HPLC (CLND):

| Composition of product | [in % by weight] |
|---|---|
| Inventive triazine compound | 100.0 |
| (as per formula (1) with | |
| R₁ as per formula (7b) and | |
| R₂ with E = —NH— and n = 5) | |
| R₂ with m = 0; | 89.8 |
| R₃ = R₂, where E = —NH—, | |
| n = 5 and m = 0 | |
| R₂ with m = 1-3; | 8.9 |
| R₃ = R₂, where E = —NH—, | |
| n = 5 and m = 0-3 | |
| R₂ with m = 0-1; | 1.3 |
| R₃ = —OH | |

### EXAMPLE 5

### Preparation of N,N′-[6-(N-[3-(diethylamino)-1-propylamino])-1,3,5-triazine-2,4-diyl]bis(6-aminohexanoic acid)

### 5.1 Preparation of Intermediate N,N′-[6-chloro-1,3,5-triazine-2,4-diyl]bis(6-aminohexanoic acid)

see Example 4.1

### 5.2 Preparation of Crude Product

186.9 g of the intermediate prepared as described in Example 5.1 (0.5 mol) were reacted in a 2 l glass reaction flask in 1 l of water with 65.1 g of 3-diethylamino-1-propylamine (0.5 mol) at 25 to 40° C. and with 80.0 g of 25% by weight aqueous sodium hydroxide solution (corresponding to 0.5 mol of sodium hydroxide) at 35 to 40° C. and heated at 101 to 103° C. for 3 hours. 1332 g of filtrate were obtained after cooling and depressurizing.

### 5.3 Work-Up of Crude Product

The filtrate was desalted and purified through 1000 ml of Amberlyst 35 (1.9 mol H⁺) similarly to Example 1.3.b). The yield was 222.4 g (0.48 mol, 95% based on amount of intermediate 5.1 used).

### Analysis of Product by HPLC (CLND):

| Composition of product | [in % by weight] |
|---|---|
| Inventive triazine compound | 100.0 |
| (as per formula (1) with | |
| R₁ as per formula (7c) and | |
| R₂, with E = —NH— and n = 5) | |
| R₂ with m = 0; | 91.7 |
| R₃ = R₂, where E = —NH—, | |
| n = 5 and m = 0 | |
| R₂ with m = 1-3; | 6.9 |
| R₃ = R₂, where E = —NH—, | |
| n = 5 and m = 0-3 | |
| R₂ with m = 0-1; | 1.4 |
| R₃ = —OH | |

### EXAMPLE 6

### Preparation of N-[4,6-bis(N-[2,2,6,6-tetramethyl-4-piperidylamino])-1,3,5-triazin-2-yl]-6-aminohexanoic acid

### 6.1-6.2 Preparation of Crude Product

In a 1 l glass flask, 46.1 g of cyanuric chloride (0.25 mol) in 300 ml of xylene (mixed isomers) are reacted with 78.2 g of 4-amino-2,2,6,6-tetramethylpiperidine (0.5 mol) at 5 to 20° C. and with 80.0 g of 25% by weight aqueous sodium hydroxide solution (corresponding to 0.5 mol of sodium hydroxide) at 20 to 25° C. and stirred at 60° C. for one hour. Concurrently, 56.6 g of ε-caprolactam (0.5 mol) were reacted with 40.0 g of 25% by weight aqueous sodium hydroxide solution (corresponding to 0.25 mol of sodium hydroxide) at 120° C. in a 250 ml glass flask for one hour, and the molten solution of aminocaproic acid and ε-caprolactam was introduced into the two-phase suspension. This reaction mixture was heated under reflux and water was azeotropically distilled off via a Dean and Stark apparatus up to a reaction temperature of 130° C. and the reaction mixture was heated at 127 to 134° C. under atmospheric pressure for a further 3 hours. After cooling to 100° C., 250 ml of water were added and the mixture was refluxed for 1 hour. At 25° C., the two-phase suspension was filtered and the phases were separated. The organic phase can be reused in a subsequent batch. 466 g were obtained of aqueous phase.

6.3 Work-Up of Crude Product The filtrate was desalted and purified through 1040 ml of Amberlyst 35 (1.96 mol H⁺) similarly to Example 1.3.b). The yield was 122.7 g (0.23 mol, 95% based on amount of cyanuric chloride used).

### Analysis of Product by HPLC (CLND):

| Composition of product | [in % by weight] |
|---|---|
| Inventive triazine compound | 100.0 |
| (as per formula (1) with | |
| R₁ as per formula (7b) and | |
| R₂ with E = —NH— and n = 5) | |
| R₂ with m = 0, R₃ = R₁ | 92.0 |
| R₂ with m = 1-3, R₃ = R₁ | 8.0 |

### EXAMPLE 7

### Preparation of N-[4,6-bis(N-[2,2,6,6-tetramethyl-4-piperidylamino])-1,3,5-triazin-2-yl]-6-aminohexanoic acid

### 7.1-7.2 Preparation of Crude Product

In a 2 l Hastelloy autoclave, 46.1 g of cyanuric chloride (0.25 mol) in 480 ml of water were reacted with 78.2 g of 4-amino-2,2,6,6-tetramethylpiperidine (0.5 mol) at 5 to 20° C. and with 80.0 g of 25% by weight aqueous sodium hydroxide solution (corresponding to 0.5 mol of sodium hydroxide) at 20 to 25° C. This suspension was stirred at 60° C. for one hour. Concurrently, 56.6 g of ε-caprolactam (0.5 mol) were reacted with 40.0 g of 25% by weight aqueous sodium hydroxide solution (corresponding to 0.25 mol of sodium hydroxide) at 120° C. in a 250 ml glass flask for one hour, and the molten solution of aminocaproic acid and ε-caprolactam was added to the aqueous suspension. The autoclave was sealed and heated for 4 hours at 153° C. and 6.1 bar. The suspension was filtered after cooling and depressurizing to obtain 778 g of filtrate.

### 7.3 Work-Up of Crude Product

The filtrate was desalted and purified through 1040 ml of Amberlyst 35 (1.96 mol H⁺) similarly to Example 1.3.b). The yield was 112.2 g (0.22 mol, 87% based on amount of cyanuric chloride used).

### Analysis of Product by HPLC (CLND):

| Composition of product | [in % by weight] |
|---|---|
| Inventive triazine compound | 100.0 |
| (as per formula (1) with | |
| R₁ as per formula (7b) and | |
| R₂ with E = —NH—, n = 5 and m = 0) | |
| R₃ = R₁ | 94.0 |
| R₃ = —OH | 6.0 |

### EXAMPLE 8

### Preparation of N-[4,6-bis(N-[2,2,6,6-tetramethyl-4-piperidylamino])-1,3,5-triazin-2-yl]-6-aminohexanoic acid

### 8.1-8.2 Preparation of Crude Product

46.1 g of cyanuric chloride (0.25 mol) in 300 ml of water were reacted with 78.2 g of 4-amino-2,2,6,6-tetramethylpiperidine (0.5 mol) and 80.0 g of 25% by weight aqueous sodium hydroxide solution (corresponding to 0.5 mol of sodium hydroxide) in a 1 l Hastelloy autoclave similarly to Example 7. Following addition of 56.6 g of ε-caprolactam (0.5 mol) and 40.0 g of 25% by weight aqueous sodium hydroxide solution (corresponding to 0.25 mol of sodium hydroxide) at 25° C., the autoclave was sealed and heated for 1 hour at 115° C. and 1.5 bar and then for 4 hours at 149° C. and 5.9 bar. The suspension was filtered after cooling and depressurizing to obtain 599 g of filtrate.

### 8.3 Work-Up of Crude Product

The filtrate was desalted and purified through 1040 ml of Amberlyst 35 (1.96 mol H⁺) similarly to Example 1.3.b). The yield was 118.4 g (0.23 mol, 91% based on amount of cyanuric chloride used).

### Analysis of Product by HPLC (CLND):

| Composition of product | [in % by weight] |
|---|---|
| Inventive triazine compound | 100.0 |
| (as per formula (1) with | |
| R₁ as per formula (7b) and | |
| R₂ with E = —NH—, n = 5 and m = 0) | |
| R₃ = R₁ | 98.0 |
| R₃ = —OH | 2.0 |

### EXAMPLE 9

### Preparation of N-[4,6-bis(N-[3-(diethylamino)-1-propylamino])-1,3,5-triazin-2-yl]-6-aminohexanoic acid

### 9.1-9.3 Preparation and Work-Up of Crude Product

46.1 g of cyanuric chloride (0.25 mol) in 300 ml of water were reacted with 65.1 g of 3-(diethylamino)-1-propylamine (0.5 mol) and 80.0 g of 25% by weight aqueous sodium hydroxide solution (corresponding to 0.5 mol of sodium hydroxide) in a 1 l Hastelloy autoclave similarly to Example 8. Following addition of 56.6 g of ε-caprolactam (0.5 mol) and 40.0 g of 25% by weight aqueous sodium hydroxide solution (corresponding to 0.25 mol of sodium hydroxide), the autoclave was sealed and heated for 1 hour at 115° C. and 1.5 bar and then for 4 hours at 15° C. and 6.0 bar. The reaction solution (588 g) was desalted and purified by ion exchange chromatography similarly to Example 8. The yield was 118.4 g (0.23 mol, 93% based on amount of cyanuric chloride used).

### Analysis of Product by HPLC (CLND):

| Composition of product | [in % by weight] |
|---|---|
| Inventive triazine compound | 100.0 |
| (as per formula (1) with | |
| R₁, as per formula (7c) and | |
| R₂ with E = —NH—, m = 0 and n = 5) | |
| R₃ = R₁ | 96.8 |
| R₃ = —OH | 3.2 |

### EXAMPLE 10

### Preparation of N-[4,6-bis(N-[2,2,6,6-tetramethyl-4-piperidyl]-N-butylamino)-1,3,5-triazin-2-yl]-6-aminohexanoic acid

46.1 g of cyanuric chloride (0.25 mol) in 300 ml of water were reacted with 106.2 g of 4-N-butylamino-2,2,6,6-tetramethylpiperidine (0.5 mol) and 80.0 g of 25% by weight aqueous sodium hydroxide solution (corresponding to 0.5 mol of sodium hydroxide) in a 1 l Hastelloy autoclave similarly to Example 8. Following addition of 56.6 g of ε-caprolactam (0.5 mol) and 40.0 g of 25% by weight aqueous sodium hydroxide solution (corresponding to 0.25 mol of sodium hydroxide), the autoclave was sealed and heated for 1 hour at 116° C. and 1.5 bar and then for 4 hours at 152° C. and 6.1 bar. The reaction solution (629 g) was desalted and purified by ion exchange chromatography similarly to Example 8. The yield was 138.2 g (0.22 mol, 88% based on amount of cyanuric chloride used).

### Analysis of Product by HPLC (CLND):

| Composition of product | [in % by weight] |
|---|---|
| Inventive triazine compound | 100.0 |
| (as per formula (1) with | |
| R₁, as per formula (7a) and | |
| R₂ with E = —NH—, m = 0 and n = 5) | |
| R₃ = R₁ | 98.0 |
| R₃ = —OH | 2.0 |

### EXAMPLE 11

### Preparation of N,N′-[6-(N-[2,2,6,6-tetramethyl-4-piperidylamino])-1,3,5-triazine-2,4-diyl]bis(6-aminohexanoic acid)

In a 1 l Hastelloy autoclave, 56.6 g of ε-caprolactam (0.5 mol) and 80.0 g of 25% by weight aqueous sodium hydroxide solution (corresponding to 0.5 mol of sodium hydroxide) were reacted at 112 to 118° C. for one hour, diluted with 300 ml of water, cooled down to 10° C., reacted with 46.1 g of cyanuric chloride (0.25 mol) at 10 to 20° C. and stirred at 20 to 25° C. for one hour. Following addition of 39.1 g of 4-amino-2,2,6,6-tetramethylpiperidine (0.25 mol) and 40.0 g of 25% by weight aqueous sodium hydroxide solution (corresponding to 0.25 mol of sodium hydroxide), the autoclave was sealed and heated for 6 hours at 150° C. and 6.0 bar.

The reaction solution (562 g) was desalted and purified by ion exchange chromatography similarly to Example 8. The yield was 108.8 g (0.22 mol, 86% based on amount of cyanuric chloride used).

### Analysis of Product by HPLC (CLND):

| Composition of product | [in % by weight] |
|---|---|
| Inventive triazine compound | 100.0 |
| (as per formula (1) with | |
| R₁ as per formula (7b) and | |
| R₂ with E = —NH— and n = 5) | |
| R₂ with m = 0; | 95.8 |
| R₃ = R₂, where E = —NH—, | |
| m = 0 and n = 5 | |
| R₂ with m = 1-3; | 3.4 |
| R₃ = R₂, where E = —NH—, | |
| m = 1-3 and n = 5 | |
| R₂ with m = 0; | 0.8 |
| R₃ = —OH | |

### EXAMPLE 12

### Preparation of N,N′-[6-(N-[3-(diethylamino)-1-propylamino])-1,3,5-triazine-2,4-diyl]bis(6-aminohexanoic acid)

In a 1 l Hastelloy autoclave, 56.6 g of ε-caprolactam (0.5 mol) and 80.0 g of 25% by weight aqueous sodium hydroxide solution (corresponding to 0.5 mol of sodium hydroxide) were reacted at 112 to 118° C. for one hour, diluted with 300 ml of water, reacted with 46.1 g of cyanuric chloride (0.25 mol) at 10 to 20° C. and stirred at 20 to 25° C. for one hour similarly to Example 11. Following addition of 32.6 g of 3-(diethylamino)-1-propylamine (0.25 mol) and 40.0 g of 25% by weight aqueous sodium hydroxide solution (corresponding to 0.25 mol of sodium hydroxide), the autoclave was sealed and heated for 6 hours at 150° C. and 6.0 bar. The reaction solution (555 g) was desalted and purified by ion exchange chromatography similarly to Example 8. The yield was 104.0 g (0.22 mol, 88% based on amount of cyanuric chloride used).

### Analysis of Product by HPLC (CLND):

| Composition of product | [in % by weight] |
|---|---|
| Inventive triazine compound | 100.0 |
| (as per formula (1) with | |
| R₁ as per formula (7c) and | |
| R₂, R₃ with E = —NH— and n = 5) | |
| R₂ with m = 0; | 96.2 |
| R₃ = R₂, where E = —NH—, | |
| m = 0 and n = 5 | |
| R₂ with m = 1-3; | 3.3 |
| R₃ = R₂, where E = —NH—, | |
| m = 1-3 and n = 5 | |
| R₂ with m = 0; | 0.5 |
| R₃ = —OH | |

### EXAMPLE 13

### Preparation of N,N′-[6-(N-[2,2,6,6-tetramethyl-4-piperidyl]-N-butylamino)-1,3,5-triazine-2,4-diyl]bis(6-aminohexanoic acid)

In a 1 l Hastelloy autoclave, 56.6 g of ε-caprolactam (0.5 mol) and 80.0 g of 25% by weight aqueous sodium hydroxide solution (corresponding to 0.5 mol of sodium hydroxide) were reacted at 112 to 118° C. for one hour, diluted with 300 ml of water, reacted with 46.1 g of cyanuric chloride (0.25 mol) at 10 to 20° C. and stirred at 20 to 25° C. for one hour similarly to Example 11. Following addition of 53.1 g of 4-N-butylamino-2,2,6,6-tetramethylpiperidine (0.25 mol) and 40.0 g of 25% by weight aqueous sodium hydroxide solution (corresponding to 0.25 mol of sodium hydroxide), the autoclave was sealed and heated for 6 hours at 150° C. and 6.0 bar. The reaction solution (578 g) was desalted and purified by ion exchange chromatography similarly to Example 8. The yield was 124.4 g (0.23 mol, 91% based on amount of cyanuric chloride used).

### Analysis of Product by HPLC (CLND):

| Composition of product | [in % by weight] |
|---|---|
| Inventive triazine compound | 100.0 |
| (as per formula (1) with | |
| R₁ as per formula (7a) and | |
| R₂ with E = —NH— and n = 5) | |
| R₂ with m = 0; | 94.9 |
| R₃ = R₂, where = —NH—, | |
| m = 0 and n = 5 | |
| R₂ with m = 1-3; | 4.1 |
| R₃ = R₂, where E = —NH—, | |
| m = 1-3 and n = 5 | |
| R₂ with m = 0; | 1.0 |
| R₃ = —OH | |

### EXAMPLE 14

### Preparation of N-[4,6-bis-(N-[2,2,6,6-tetramethyl-4-piperidylamino])-1,3,5-triazin-2-yl]-6-aminohexanoic acid

### 14.1-14.2 Preparation of Crude Product

In a 1 l Hastelloy autoclave, 46.1 g of cyanuric chloride (0.25 mol) in 300 ml of water were reacted with 78.2 g of 4-amino-2,2,6,6-tetramethylpiperidine (0.5 mol) and with 80.0 g of 25% by weight aqueous sodium hydroxide solution (corresponding to 0.5 mol of sodium hydroxide) at 20 to 25° C. similarly to Example 7. This suspension was stirred at 60° C. for one hour. Concurrently, 54.3 g of ε-laurolactam (0.28 mol) were reacted with 40.0 g of 25% by weight aqueous sodium hydroxide solution (corresponding to 0.25 mol of sodium hydroxide) at 120° C. in a 250 ml glass flask for one hour, and the molten solution of 1-aminododecanoic acid and ε-laurolactam was added to the aqueous suspension. The autoclave was sealed and heated for 4 hours at 153° C. and 6.1 bar. The suspension was filtered after cooling and depressurizing to obtain 780 g of filtrate.

### 14.3 Work-Up of Crude Product

The filtrate was desalted and purified through 1040 ml of Amberlyst 35 (1.96 mol H⁺) similarly to Example 1.3.b). The yield was 129.3 g (0.21 mol, 86% based on amount of cyanuric chloride used).

### Analysis of Product by HPLC (CLND):

| Composition of product | [in % by weight] |
|---|---|
| Inventive triazine compound | 100.0 |
| (as per formula (1) with | |
| R₁ as per formula (7b) and | |
| R₂ with E = —NH— and | |
| n = 11 and m = 0) | |
| R₃ = R₁ | 95.2 |
| R₃ = —OH | 4.8 |

1. A triazine compound of the formula 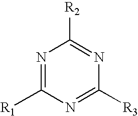
where
R₁ is -A-B
with A is —O— or —NR₄—,
B is amino-containing substituent and
R₄ is hydrogen or alkyl
R₂ is 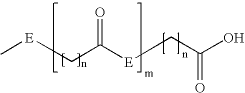
where
E is —O— or —NR₅—,
n is 3 to 15,
m is 0 to 10 and
R₅ is hydrogen or alkyl
R₃ is R₁, R₂, —OR₆ or —NR₇R₈
where R₆, R₇ and R₈ is hydrogen, alkyl or aryl, each substituted or unsubstituted.

2. The triazine compound according to claim 1, wherein the triazine compound comprises an R₁ substituent having a B substituent which conforms to the formula 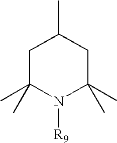
where
R₉ is hydrogen, alkyl or alkoxy of the formula —O—R₂₀,
R₂₀ is a branched or unbranched alkyl or cycloalkyl group having in either case from 4 to 16 carbon atoms,
or which conforms to the formula
—(CH₂)ₚ—NR₁₀R₁₁
where p is from 1 to 15 and
R₁₀, R₁₁ is hydrogen, alkyl, cycloalkyl or heterocycloalkyl and R₁₀ and R₁₁ are the same or different.

3. The triazine compound according to claim 2, wherein the triazine compound comprises an R₁ substituent conforming to the formula 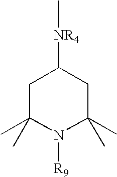
where
R₉ is hydrogen, alkyl or alkoxy of the formula —O—R₂₀,
R₂₀ is a branched or unbranched alkyl or cycloalkyl group having in either case from 4 to 16 carbon atoms,
R₄ is hydrogen or alkyl.

4. The triazine compound according to claim 1, wherein the triazine compound comprises an R₁ substituent as R₃ substituent.

5. The triazine compound according to claim 1, wherein the triazine compound comprises an R₂ substituent as R₃ substituent.

6. A process for preparing a triazine compound of the formula 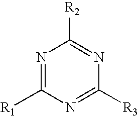
where
R₁ is -A-B
with
A is —O— or —NR₄—,
B is amino-containing substituent and
R₄ is hydrogen or alkyl
R₂ is 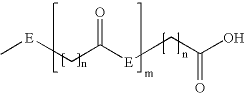
where
E is —O— or —NR₅—,
n is 3 to 15,
m is 0 to 10 and
R₅ is hydrogen or alkyl
R₃ is R₁, R₂, —OR₆ or —NR₇R₈
where R₆, R₇ and R₈ is hydrogen, alkyl or aryl, each substituted or unsubstituted, comprising:
(1) reacting cyanuric chloride is reacted with from 0.5 to 5 mole equivalents of an amine of the formula
H-A-B
in the presence of a base to form an intermediate, which is further reacted with from 0.5 to 5 mole equivalents of a compound conforming to the formula 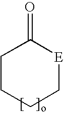
or of a compound conforming to the formula 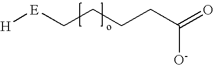
where
o is from 0 to 12,
E is —O— or —NR₅— and
R₅ is hydrogen or alkyl,
or
(2) reacting cyanuric chloride with from 0.5 to 5 mole equivalents of a compound conforming to the formula 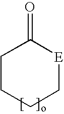
or of a compound conforming to the formula 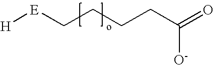
where
o is from 0 to 12,
E is —O— or —NR₅— and
R₅ is hydrogen or alkyl,
in the presence of a base to form an intermediate which is further reacted with from 0.5 to 5 mole equivalents of an amine of the formula
H-A-B
to obtain said triazine.

7. The process according to claim 6, wherein, in a further reaction step, 0.5 to 5 mole equivalents of a compound conforming to the formulae
H—OR₆
or
H—NR₇R₈
are reacted in the presence of a base.

8. A composition comprising, at least two different triazine compounds conforming to the formula 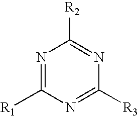
where
R₁ is -A-B
with
A is —O— or —NR₄—,
B is amino-containing substituent and
R₄ hydrogen or alkyl
R₂ is 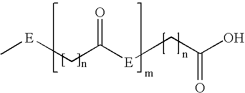
where
E is —O— or —NR₅—,
n is 3 to 15,
m is 0 to 10 and
R₅ is hydrogen or alkyl
R₃ is R₁, R₂, —OR₆ or —NR₇R₈
where R₆, R₇ and R₈ is hydrogen, alkyl or aryl, each substituted or unsubstituted.

9. A solution comprising, at least one triazine compound conforming to the formula 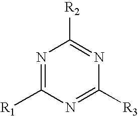
where
R₁ is -A-B
with
A is —O— or —NR₄—,
B is amino-containing substituent and
R₄ is hydrogen or alkyl
R₂ is 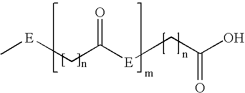
where
E is —O— or —NR₅—,
n is 3 to 15,
m is 0 to 10 and
R₅ is hydrogen or alkyl
R₃ is R₁, R₂, —OR₆ or —NR₇R₈
where R₆, R₇ and R₈ is hydrogen, alkyl or aryl, each substituted or unsubstituted.

10. The solution according to claim 9, wherein the solution comprises water as solvent.

11. The solution according to claim 9, wherein the solution comprises from 1% to 50% by weight of triazine compounds.
